(19) United States
(12) Patent Application Publication
(10) Pub. No.: US 2004/0073094 A1
(43) Pub. Date: Apr. 15, 2004

(54) Fetal monitoring systems with ambulatory patient units and telemetric links for improved uses

(76) Inventor: Donald A. Baker, Colbert, WA (US)

Correspondence Address: RANDY A. GREGORY, P.O BOX 31090, SPOKANE, WA, 99223-3018, US

(21) Appl. No.: 10/271,890

(22) Filed: Oct. 15, 2002

(51) Int. Cl. A61B 5/00

(52) U.S. Cl. 600/300

(57) ABSTRACT

A fetal monitoring system, preferably with telemetric links, to communicate fetal health information from an expectant woman to a receiving station. The monitoring units can be self-installed by the patient and worn in a reception or other monitoring room properly equipped to receive the information telemetrically. Other systems of the invention include a visual display which is used to present educational material to the expectant mother or other patient during monitoring. A doctor, nurse or an automated testing routine can be used to confirm delivery of the educational course and/or treatment information. Preferred systems allows prenatal or other medical education to be given, documented, and can also provide automated verification to help prevent billing abuse.

### TECHNICAL FIELD

The technical field of this invention is fetal monitoring apparatus and related methods and systems for improved medical services, patient monitoring, and medical records keeping.

### BACKGROUND OF THE INVENTION

One of the frequently used techniques for monitoring fetal health uses ultrasound imaging. This requires a relatively costly piece of ultrasound imaging equipment. Ultrasound evaluations also typically involve nurses or doctors functioning as the examiner or attending medical personnel. Accordingly, this procedure has significant associated service charges to reflect both the equipment costs and the costly medical services personnel used.

The use of ultrasound imaging techniques for fetal monitoring or diagnostic testing involves beaming an ultrasonic beam through the abdomen of a pregnant woman. A pregnant or gravid woman is also referred to as a gravida. The ultrasonic beam is directed upon the fetus and reflected ultrasonic waves are produced. The reflected ultrasonic waves are sensed and used to produce an image of the fetus. Although this is a relatively common procedure, it involves using a relatively high energy ultrasonic beam to provide better reflected signals from which the image of the fetus is compiled.

Imaging the fetus using ultrasound has strong appeal and interest for the expectant mother and other family members. Although ultrasound imaging is of interest in a number of situations, it is not an efficient and economical approach for widespread routine fetal monitoring. Accordingly, medical service providers are in need of more efficient monitoring systems and procedures for routine determination of fetal well-being.

Some medical service providers want the popular demand for ultrasound imaging of fetuses to be paid by those patients interested in such services, such as on an optional basis. Others tend to discourage such ultrasound imaging procedures to save money, thus leaving the fetus without routine monitoring except using a stethoscope or equivalent. This underscores the long felt need for low cost, routine fetal monitoring and analysis which is convenient, reliable and provides greater diagnostic ability than stethoscopes or their equivalents.

Another fetal monitoring technique sometimes used employs Doppler ultrasound. In Doppler ultrasound examinations, the equipment detects moving blood flow. This is typically used to detect blood flow in the umbilical cord or the fetal heart and other fetal blood vessels. The technique is most frequently used to determine fetal cardiac function, particularly fetal heart rate.

Although ultrasound examination of fetuses is common practice, there are some who believe beaming of ultrasound may pose risks to the fetus. Current medical knowledge has not definitively answered this question. Nonetheless, there is some impetus to reduce exposure of fetuses to ultrasound unless the appropriateness of ultrasound is clearly indicated. Use of ultrasound imaging may be indicated when an acute symptom or symptoms are present suggesting fetal or maternal distress.

One routine fetal health test is commonly called the non-stress test. In this test the fetus is felt, detected, observed or otherwise noticed moving in the womb as a result of normal fetal movements. Such fetal movements necessarily involve the expenditure of effort by the fetus. After movement is noticed, then there should be an associated change in the fetal heart rate. This fetal heart rate change is typically a moderate increase. The observed fetal heart rate change should be within an acceptable range. If the fetal heart rate change is not within an acceptable range, then fetal health problems are indicated. Little or no increase in fetal heart rate, or an excessive fetal heart rate increase, are both indicative of possible fetal health problems.

Because current ultrasound fetal monitoring procedures are relatively costly to perform on a routine basis, this is of particular concern to insurance companies and government health care agencies. These organizations pay large portions of the total costs of health care. Accordingly, health insurance companies and/or government health care agencies are often reluctant to pay for routine fetal ultrasound examinations. This is an issue because of the frequent demand for this service and the significant costs. Accordingly, such institutions often deny or limit benefits for this service, unless it is needed for specific medical concerns or demonstrable health problems.

Another consideration relating to the reluctance of health insurers and government agencies is the pressure applied by family members seeking to have physicians use ultrasound for fetal examinations. Such is often sought for determining the sex of an expected child, or for routine interest of family members to make sure the fetus looks healthy. These justifications are viewed with skepticism by the organizations paying for such services. Accordingly, more economical procedures and equipment are needed to allow regular monitoring of fetal health. In particular, improvements are needed for conducting routine non-stress tests or other analyses of fetal health on an economical and widespread basis.

In the area of fetal health monitoring and prenatal care there is also a particular need for the expectant mother to be educated about prenatal health practices and symptoms. In many instances pregnant women may not have a good understanding of either risky behavior or symptoms to watch for during the course of a pregnancy. This applies to health practices of the woman as well as symptoms which directly indicate fetal health problems. Risky behavior patterns demonstrated by some pregnant women may be a factor in bringing about fetal health distress and/or birth defects. Thus, it is desirable for prenatal medical education to be communicated as effectively and efficiently as possible to help make pregnant women aware of fetal health symptoms and better prenatal health practices.

Medical education of patients is also important because patients sometimes accuse doctors of not adequately informing them about health risks associated with an observed medical condition or treatment approach. This also applies to informing patients about the risks associated with medical treatments and the likelihood of achieving successful results from a treatment procedure. This is made even more difficult where there are plural alternative treatments each having different risks and benefits.

These risks sometimes result in malpractice claims being made against doctors or other health care providers. Such potential claims are a particular problem for doctors involved with obstetrics and surgery. However, the problem also exists generally for all health care personnel and health care service organizations.

In some malpractice cases the development of a patient health problem may be exploited by a person with faulty memory or deceptive intent to collect on a malpractice claim. This may be successful merely because there is insufficient or no documentation proving that adequate information was given to a patient before the patient or other responsible person consented to medical treatment.

Alternatively, in some cases there may be insufficient documentation showing a person who refuses treatment was informed about risks associated with not receiving treatment. Documenting patient education and other cautionary information given by a medical service provider to pregnant women or other patients may be important in defeating or mitigating medical malpractice claims in a variety of situations.

There are also many instances where medical services funded by health insurance companies or government health care agencies are provided in a manner designed to abuse billing practices. In some cases doctors, hospitals or other health care providers may not render services or may render abbreviated services and bill the funding organization. Accordingly, there is a need for improved service, accounting and auditing practices which are performed and organized in a manner which reduces the potential or extent of abuse which may occur.

These and other problems, considerations, objectives and benefits are at least in part pertinent to various aspects of the inventions described herein. There may be additional benefits, advantages or principals of operation concerning the inventions which may not be recognized or fully understood or appreciated at this time. These may come to light later and be significant in considering the issues relating to patentability. The best mode of the invention as currently known is described herein.

### BRIEF DESCRIPTION OF THE DRAWINGS

Preferred embodiments of the invention are described below with reference to the accompanying drawings, which are briefly described below.

FIG. 1 is a perspective view of a doctor's office reception room fitted with a preferred system according to the invention.

FIG. 2 is a diagram representing a cross-sectional view through a pregnant woman illustrating some aspects of the invention and how it is installed upon a pregnant patient.

FIG. 3 is a perspective diagrammatic view showing another embodiment of the invention installed and used in a home or other location.

FIG. 4 is a perspective view showing another embodiment of the invention installed and used in a space vehicle.

FIG. 5 is a perspective view illustrating another embodiment wherein a pregnant woman is in a special unit constructed in accordance with the invention.

FIG. 6 is a perspective view illustrating a further embodiment wherein a pregnant woman is seated in a chair using a portion of one system made in accordance with the invention.

FIG. 7 is a side view showing a preferred sensor construction according hereto.

FIG. 8 is a front view of the sensor construction of FIG. 7.

FIG. 9 is a front view with portions removed of the sensor construction of FIG. 7.

FIG. 10 is a cross-sectional view of the sensor of FIG. 7 taken along line 10-10 of FIG. 9.

FIG. 11 is a perspective view of a contact piece in isolation which forms part of the sensor construction of FIG. 7.

FIG. 12 is a block diagram illustrating a first portion of preferred aspects and procedures in accordance with the invention.

FIGS. 13 is a block diagram illustrating a second portion relating to the first portion of FIG. 12.

FIG. 14 is a block diagram illustrating preferred procedures for educating, testing and validating services for payment.

### DETAILED DESCRIPTION OF THE PREFERRED EMBODIMENTS

● Explanation Concerning Terminology ●

The embodiments described herein may rely on terminology used in any section of this document and other terms known in the related technology areas. This document is premised upon using one or more terms with one embodiment that will in general apply to other embodiments for similar structures, functions, features and aspects of the invention. Wording used in the claims is also descriptive of the invention. Terminology used with one, some or all embodiments may be used for describing and defining the technology and exclusive rights associated herewith.

● General System and Associated Monitoring Room ●

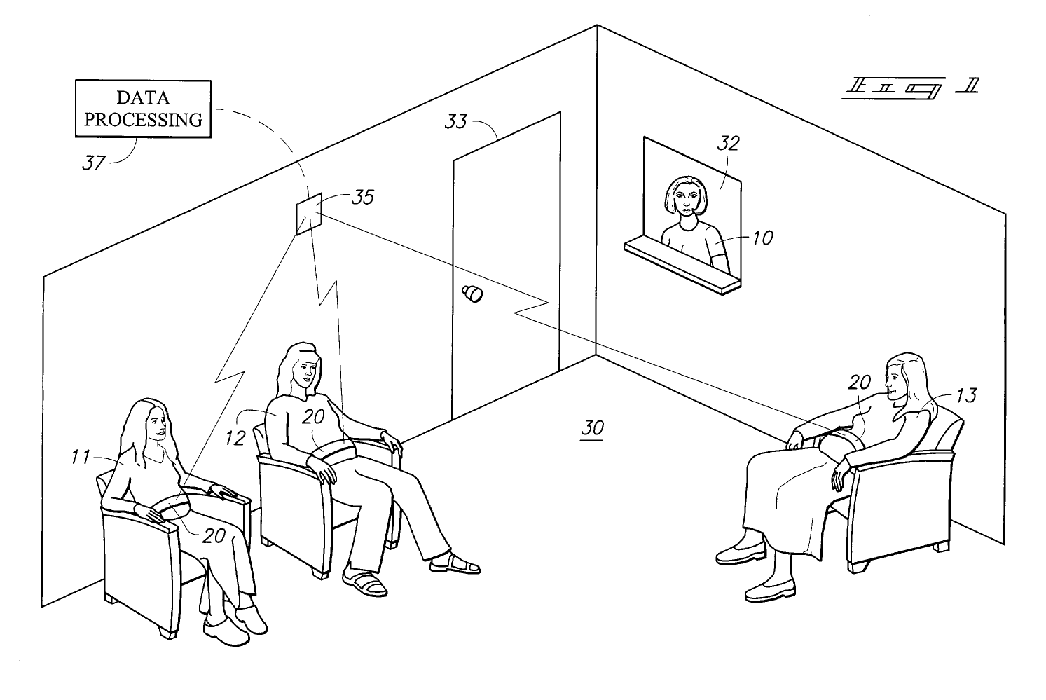

A preferred system according to the present invention is generally shown by FIG. 1. The system is implemented in a receiving or waiting area 30 of a physician's office or in another similar type of room adapted to provide the desired features discussed below. The waiting or other monitoring area 30 may also be in medical services facilities operated by hospitals, public health facilities, urgent care facilities, pharmacies or in other locations used by medical service providers. Another alternative of particular interest is drug store space near pharmacies with pharmacists acting as the attending medical service provider.

The preferred combined waiting and monitoring area shown does not demand the costly fixtures and equipment needed or used in normal examination rooms used by physicians or in-hospital examination or patient rooms. Both of these types of facilities have plumbing, medical equipment, examination tables and other items with relatively high costs. These high costs are borne by the consumer, or medical services insurer and thus are ultimately paid by the consumer.

As shown in FIG. 1, the monitoring area is a receiving or reception area of a doctor's office which has been adapted in accordance with the invention. The room is easily staffed by a physician staff member 10 who can be a nurse or less-highly trained aid or receptionist. The receiving room is advantageously provided with a service window 32 through which the staff member 10 can dispense fetal monitoring ambulatory units 20, also called gravida units.

The reception or other monitoring room 30 is also provided with a local area communications unit 35 which is in communication with the fetal monitoring units 20. The communications unit can be used to send and/or receive information with the monitoring units and provide data collection therefrom, as explained more fully below. In some of the preferred embodiments, the local area communications unit 35 is thus telemetrically linked to the ambulatory fetal monitoring units 20 to advantageously allow movement of the patients without impedance from wires. Some forms of the invention may be implemented using wired communications links, although wireless or telemetric linking is preferred.

The communications unit 35 is preferably connected to a computer or other data processing device 37 which allows the data to be conveniently stored and analyzed. The data processing unit is preferably located outside of the monitoring room 30, such as in a secured data processing area. A variety of data processing units may be employed as unit 37, such as commonly used for desktop computers, file servers, main frame computers and other types of suitable data processing apparatuses.

The monitoring room is advantageously provided with seating for use by patients 11, 12 and 13. These patients are obstetric patients who have ambulatory monitoring units 20 thereon. The monitoring units 20 can be self-installed by the patients to reduce medical staff workload. Alternatively, for new patients or those having troubles, the attending staff member 10 may assist in installing or applying the ambulatory fetal monitoring units 20 upon the patients.

FIG. 1 also shows a door 33 which controls access and movement of patients from the reception area or other monitoring room to other areas of the doctor's office, pharmacy or other medical facility. The fetal monitoring room 30 allows routine fetal monitoring and related educational and testing services to be performed within a relatively low cost area. This arrangement and approach frees normal patient examination rooms for use in serving patients that require a private examination room or other room having specialized equipment.

● Ambulatory Fetal Monitoring Gravida Units ●

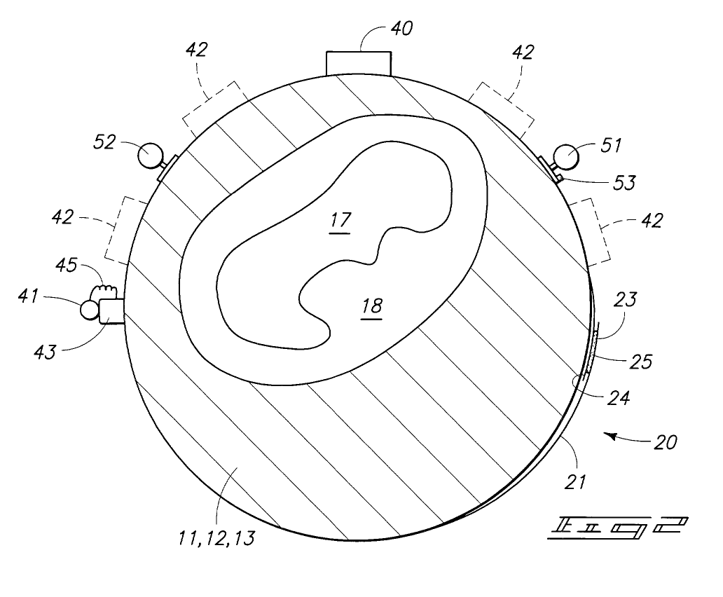

FIG. 2 shows a diagram representing a cross-sectional view of a pregnant woman with an ambulatory fetal monitoring unit 20 installed about the woman's torso 11, 12, 13. A fetus 17 within the amnion 18 is shown diagrammatically. The ambulatory fetal monitoring units preferably include one or more sensors 40, 42, 51, 52 and a sensor support 21. The sensor support holds the sensors and preferably allows the sensors to be positioned upon the gravida for detection of fetal health indicators, in particular, fetal sounds, such as fetal heart sounds, or other fetal health indicators.

The ambulatory fetal monitoring units according to this invention may also include other features and aspects as further described herein.

● ● Ambulatory Monitoring Unit Sensor Support ● ●

The ambulatory gravida unit 20 advantageously includes a belt or other sensor support 21. The belt or cummerbund, a differently configured supporting garment, or other sensor support can be made in a number of suitable configurations. As shown, the ambulatory unit has a waist or girth band designed to extend around the gravida's torso to position the sensor units appropriately upon the gravida's abdomen. The illustrated sensor support belt also has overlapping portions 23, 24 with a mating closure 25. Closure 25 can be a hook and loop closure, belt buckle, detachable clip or other suitable detachable closure constructions.

● ● Monitoring Unit Sensors ● ●

Sensor support 21 has one or more sensors 40, 42, 51, 52 mounted thereon. Sensors 40, 42, 51 and 52 are appropriately arranged about the sensor support, as needed, for proper positioning of the sensors and sensor support upon the woman's torso when worn or otherwise applied to the woman's body. The preferred positioning will depend upon the particular type of sensor or sensors being employed. The ambulatory monitoring unit may have one or plural sensors and/or detectors.

The number of sensors may depend upon the type of sensor or sensors used. It is possible to use a sensor array including a plurality of sensors. It is also possible to use multiple sensors of differing types. Alternatively, redundant sensors or sensor arrays may also be used having enhanced capabilities to sense the fetal heartbeat or other fetal indications.

Gravida unit 20 may include a single acoustic monitoring sensor 40 which is adapted to sense or detect acoustic waves developed by the fetal heart (not specifically shown). The fetal heart sounds must compete with other sounds, other acoustic waves, or other interference striking or otherwise affecting the sensor or its electrical output signal. Accordingly, the sensor or sensors or associated signal conditioning are best constructed to discriminate and selectively determine those of fetal origin, particularly of fetal cardiac origin, from background or extraneous noise and other interference.

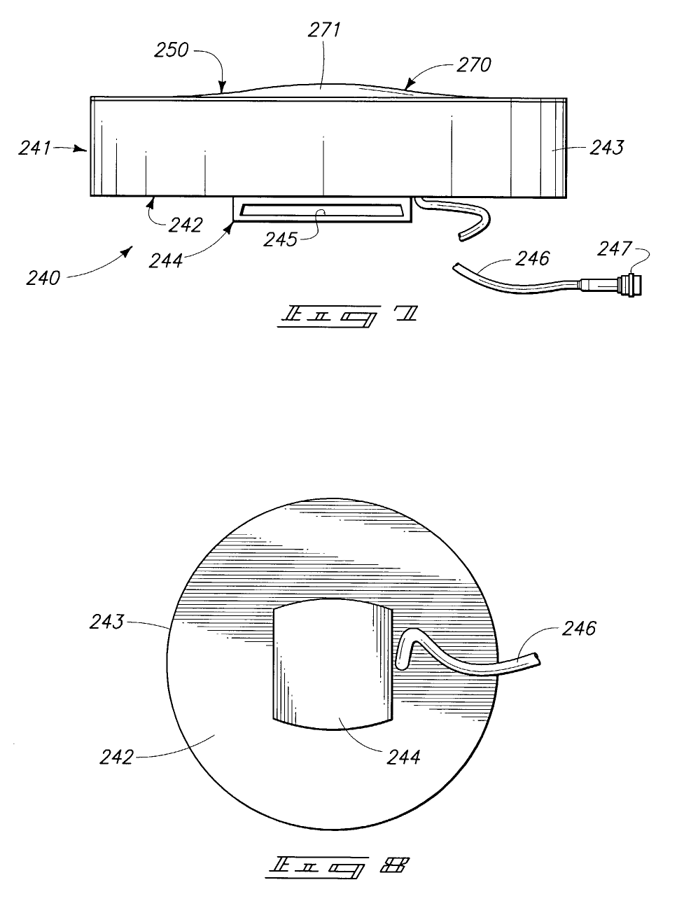

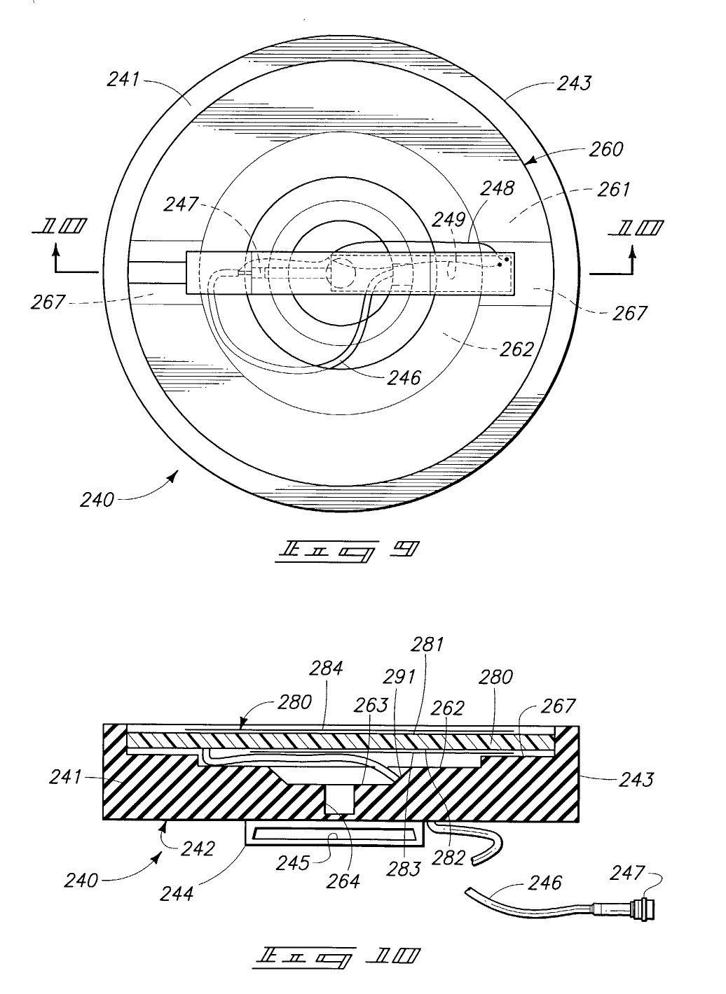

FIGS. 7-11 show one preferred sensor assembly 240 which is advantageously used as part of the gravida unit 20. This sensor assembly 240 is advantageously used for sensor 40 shown in FIG. 2. In alternative modes, sensor assembly 240 may be used to provide multiple sensor assemblies, such as sensor assemblies 42 in FIG. 2. Other mounting configurations are also possible. 20

Sensor assembly 240 has a frame or base 241 which is advantageously shaped in a generally cylindrical configuration, although other shapes or configurations are acceptable depending on other aspects of the sensor assembly. The base 241 as shown has a cylindrical side wall 243 and a distal or back face 242. The front or proximate face 250 is designed to be and is used proximate to the maternal abdomen, and will be described in greater detail below.

The assembly base has a support or mounting feature 244 which is constructed to allow a belt or other supporting member (not shown in FIGS. 7-11) to engage therewith. As shown, mounting feature 244 is in the form of a belt sleeve having a belt receiving passageway or channel 245 which extends therethrough. The belt passageway is advantageously constructed to allow a belt of nylon belting or other suitable belting materials to slide therein to allow adjustment of the sensor assembly upon the belt for changing the position of the sensor upon the gravida's abdomen.

FIG. 7 further shows a sensor assembly output signal conductor cable 246 which may be primarily or optionally used to conduct the output signal from the sensor or sensors forming part of the sensor assembly. The signal cable 246 can be electrically connected to a separate transmission unit worn on the gravida unit outside sensor assembly 240. Alternatively or optionally, the signal conductor cable 246 can be used to allow the sensor assembly to be connected by hard wiring to a diagnostic or signal processing unit (not shown), such as for maintenance evaluation or when telemetric communication is not needed or not otherwise desired. As shown, signal cable 246 has a connection terminal 247 which allows connection of the electrical conductors of signal cable 246 to an associated piece of equipment receiving the output signal or signals from sensor assembly 240. It should also be appreciated that the output signals from sensor assembly may alternatively be transmitted telemetrically from the sensor assembly to a related signal receiver, such as explained elsewhere in this document.

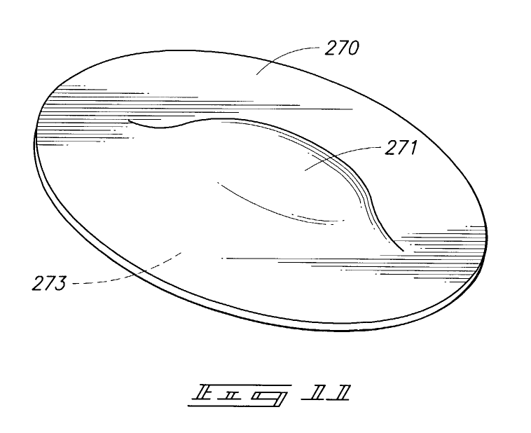

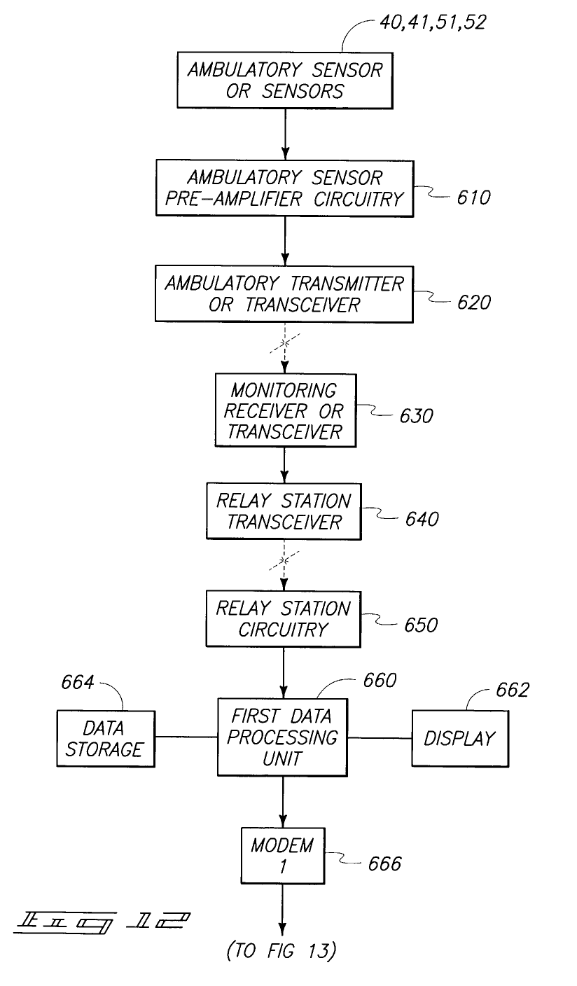

FIGS. 9 and 10 show the internal components of sensor assembly 240 in greater detail and will now be described. The base 241 has an internal cavity or receptacle 260 which is used to mount sensor elements and related components. Receptacle 260 has several sectors which include an outer annular sector or first sector 261. The first annular sector 261 is used to receive and mount a face piece 270 shown in isolation in FIG. 11. The face piece is advantageously made of a suitable soft but acoustically compliant material such as a low density polyurethane or other suitable plastics. The face piece is advantageously provided with a central dome or projection 271 which mechanically focuses the acoustical pickup towards the middle of the face piece and underlying sensor components. The back surface 273 of the face piece contacts sensor components described below to couple the acoustic waves being transmitted from the gravida's abdomen therethrough to the sensor components.

The internal cavity 260 also has a second annular sector 262 which is smaller in diameter and further recessed below the first sector 261. Second sector 262 provides space for free deformation of the sensor member 280 and allows the signal conductor cable 246 to run beneath the sensor member 280. The sensor signal cable further extend into the third annular sector 263 and passes through the base 241 at cable aperture 291.

A third annular sector 263 provides additional space for cable 246 to extend into the internal cavity and extend sufficiently far thereinto so as to be mechanically flexible and thus not interfere with the acoustic responsiveness of the sensor member 280.

A fourth annular sector 264 is of still further reduced diametrical size and greater depth. Fourth sector 264 can be used to provide additional space for running cable 246 therethrough in alternative configurations.

FIG. 9 further shows details of a preferred construction for the sensor components. The sensor components include a piezoelectric element 280 which is positioned within a channel 267 cut below the surface of first annular sector 261. The piezoelectric element can be selected from a variety of suitable piezoelectric materials, such as polyvinylidene fluoride and others.

The piezoelectric element 280 serves as a beam supported across the second annular sector with deflection occurring in response to acoustic waves transmitted through face piece 270. The face piece underside 273 bears upon an upper cover piece 284 which is electrically isolated from the piezoelectric element 280. FIG. 10 also shows the upper pole layer 281 upon the upper face of piezoelectric element 280 and the lower pole layer 282 upon the lower face of the piezoelectric element. The lower pole layer 282 is electrically isolated from a lower cover piece 283. The upper and lower cover pieces are preferably provided with conductive portions not in electrical contact with the pole of the piezoelectric material which are connected to a ground lead 247 (FIG. 9) which is connected to a separate conductor of signal cable 246. The upper pole is connected by an upper pole lead 248 to another conductor of signal cable 246, and the lower pole is connected by a lower pole lead 249 to still another conductor of signal cable 246.

Flexure of piezoelectric element 280 generates an electrical charge which is a function of the acoustic wave pattern developed therein to pick up sounds, including fetal sounds, in particular fetal cardiac sounds, so that the fetal heart rhythm can be determined and used in the analyses indicated elsewhere herein.

An alternative sensor which can be used as an acoustic sensor in gravida unit 20 is an electric microphone commercially produced and sold under the trademark and model designation EMKAY EK-3132. This acoustic sensor is an electret type having low power requirements. This unit has been used by NASA in connection with one or more space missions for voice recognition and ambient noise listening on the Mars spacecraft.

Another possible sensor was developed by NASA for use in detecting fetal sounds and is shown in U.S. Pat. No. 5,140,992 to Zuckerwar et al. U.S. Pat. No. 5,140,992 is incorporated by reference hereinto entirely. Such sensor may alternatively be useful in the inventions described herein.

It has been found that utilizing an acoustic sensor or sensors which are most responsive to sounds in the general range of 10-200 Hz is preferred. More preferably, the sensors are best adapted to sense in two different key frequency ranges. The first or lower acoustic sensor frequency detection range is about 15-60 Hz, more preferably approximately 15-40 Hz, even more preferably about 15-35 Hz. This is most sensitive when the fetus is sensed using acoustic waves traveling via fluid coupling through the amniotic fluid.

The second or upper acoustic frequency range is more preferred for use during periods where the fetus is acoustically coupled to the sensor or sensor by body tissue as compared to amniotic fluid. The preferred second or upper frequency range is approximately 60-200 Hz, more preferably, about 80-120 Hz.

As indicated above, a variety of different sensors can be used. One group or type of suitable sensors utilize piezoelectric sensor elements. These or other 20 sensors can be used in a number of different constructions and configurations in accordance with this invention.

● ● Sensor Signal Processing ● ●

To enhance the fetal heart sounds it may also be desirable to use signal processing. The preferred signal processing is used and performs by discriminating the desired fetal cardiac sounds from other sounds or sources of interference. My U.S. Pat. No. 4,781,200 issued Nov. 1, 1988 shows a digital signal processing system used to help discriminate fetal heart sounds from other sensed waveforms. U.S. Pat. No. 4,781,200 is hereby incorporated by reference entirely. Such patent discloses suitable signal processing and sensors arrangements which can be used on ambulatory fetal monitoring units according to this invention.

Another signal processing system which may be used in the inventions is shown and described in U.S. Pat. No. 5,524,631 to Zahorian which is incorporated by reference entirely. Still other commercially available signal processing systems, such as a variety of digital audio signal processing software and related implementing controllers are suitable for use herein.

● ● Multiple Sensor Array ● ●

FIG. 2 also shows an alternative sensor array using multiple sensors 42 shown in phantom. Sensors 42 are acoustic or other suitable sensors in a multi-sensor fetal sensor array. U.S. Pat. No. 4,781,200 shows a multi-sensor array which produces signals which are enhanced by a digital signal processing system having multiple channels which compare the channels to discriminate the sensor with best reception. The signal processor may utilize such sensor's signal as a primary determinate of an enhanced output signal. This approach can be used in the current inventions.

● ● Alternative Sensors ● ●

The system described in U.S. Pat. No. 4,781,200 also utilizes a sensor for detecting movement of the fetus. Although this is possible and may be preferred in some of the systems according to this invention, it is not an essential requirement in some forms of the inventions made in accordance herewith.

A number of additional sensor and signal processing designs are known and may potentially be useful in the systems according to this invention. New designs are also likely to be developed in the future which may be useful with these inventions. Acoustic sensors are preferred at this time due to their inherently passive and non-invasive nature. However, other types of sensors, such as electro-cardiographic sensors, bio-impedance sensors, and others may also be operable for use in the inventions. Signal processing for such alternative sensor is addressed by the multiplexed signal processing of the Zahorian patent referenced above.

Another suitable sensor is shown in my earlier U.S. Pat. No. 5,749,831 issued May 12, 1998 which is also incorporated by reference entirely. Such system utilizes a doppler ultrasound beam which is directed at the umbilical cord to detect umbilical blood pulsations. From this information the fetal cardiac inotropic and heart rate values are determined. Such a system may also determine other aspects indicating fetal health as described therein.

It is also possible to use a doppler ultrasound sensor subsystem in lieu of or as a secondary sensor 42 to supplement the primary acoustic sensor 40 or other types of sensors. This may be needed when the primary sensors are not able to adequately detect fetal heart beats or other detected fetal health indicators.

In a preferred configuration the gravida unit 20 uses an acoustic sensor 40 and an alternate doppler ultrasound sensor which is diagrammatically represented by sensor 41 in this alternative construction. The doppler ultrasound sensor 41 may use conventional technology, such as Parks brand OB doppler ultrasound model 115. Other such doppler ultrasound unit may also be suitable for use in the inventions. The ultrasound sensor 41 is connected to the ultrasound electronics and power unit 43 using a flexible cord 45 which allows the sensor 41 to be detached therefrom and moved to a suitable position upon the abdomen of the gravida. This configuration allows the sensor to be repositioned to various orientations and abdominal contact positions to allow the gravida to find a sensing location and orientation that results in adequate detection of the fetal cardiac functions using this type of sensor. In the preferred construction and methods the moveable doppler ultrasound sensor is only used if the passive sensor or sensors, such as acoustic sensor 40 are not picking up an adequate signal or are otherwise inoperable.

In the use of Doppler ultrasound sensor as a secondary sensor subsystem, the gravida, attending physician or other attendant can manipulate the Doppler ultrasound emitter and pickup head to better aim the ultrasound beam and provide detection in difficult situations wherein the primary sensors, such as an acoustic sensor or sensors, are not providing adequate signals for various reasons. Although the use of ultrasound is not preferred as the primary sensor, its use as a secondary sensor when other sensors are unable to perform provides improved system performance and reliability.

● ● Integrated Fetal Motion Sensing ● ●

Another alternative or optional ambulatory sensor array is used to sense fetal motion automatically, if desired and switched to be in operation. In the preferred configuration, this type of sensor array is controlled by the gravida using switch 53. Switch 53 can be manipulated to render the motion sensing function inoperable, such as when the gravida wishes to move about the room and motion sensing is prone to detecting maternal movements instead of the desired fetal movements. After the gravida has returned to a quiet location and position, then switch 53 can again be manipulated to render the motion sensing operational.

An exemplary motion sensor array of this type is shown and described in U.S. Pat. No. 4,625,733 issued Dec. 2, 1986, which is incorporated by reference entirely. FIG. 2 shows a pair of sensors 51 and 52 which are inductive sensors which emit electromagnetic waves. The sensors preferably have orthogonally oriented inductors, such as in the form of inductive coils, for emitting electromagnetic fields in a three-dimensional electromagnetic wave configuration.

The sensors 51 and 53 emit electromagnetic waves which pass to a remote sensing antenna or antennas. Movement of the fetus differentially disturbs the electromagnetic waves' comparative phase relationship. The waves are then detected and analyzed by associated sent phase comparator circuitry. The analyzed wave forms affected by the moving fetus are used to indicate fetal movement to other parts of the ambulatory gravida unit. It should also be understood that other fetal movement sensors can be used for one or both of movement sensor 51 and 52 in lieu of the sensors of the type just described. Accelerometer sensors and other types of movement sensors can be used.

● Second Embodiment System—Home Monitoring System ●

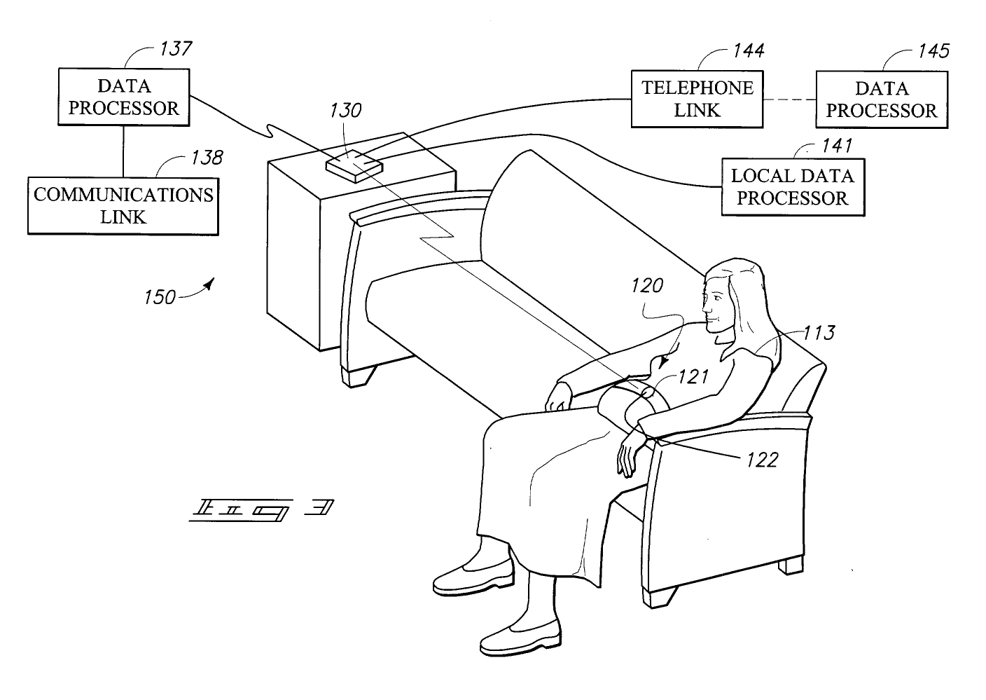

FIG. 3 shows another system 150 configured for use in helping to monitor a pregnant woman or gravida 113 to indicate potential situations where the health of the fetus or health of the gravida is of concern. This may include or require home monitoring on a continuous, nearly continuous or intermittent basis. The home system includes an ambulatory gravida unit 120 having a sensor support 121 for supporting a sensor 120 or multiple sensors and/or optional sensors in a configuration such as described above in connection with the first gravida unit 20. The ambulatory unit 120 has a transmitter (not illustrated in FIG. 3) which transmits information telemetrically to a telemetric receiver unit 130. The telemetric receiver unit 130 is preferably connected to a data processing unit 137. The data processing unit 137 can pass communications information to and/or from ambulatory unit 120 directly or indirectly to another communications link 138. Examples of such a communications link include links to a telephone or computer. Additional possible communications links may also include direct or modem connection to a global or wide area computer information communications system, such as via the internet.

FIG. 3 also illustrates additional embodiments which can be used to implement the inventions. The telemetric unit 130 may be a telemetric transceiver unit which can be directly connected for data communication to and/or from a local data processor 141 which can store data and provide operational control information to telemetric unit 130 for communication to the telemetrically linked ambulatory gravida unit 120. The local data processor may additionally perform a variety of data processing and analysis functions using data gathered by the ambulatory unit, and/or using data previously contained on the data processing unit 141 in comparison with or combination with data provided by ambulatory unit 120.

FIG. 3 still further shows another alternative arrangement wherein the telemetric unit 130 is connected to a communications link, such as the illustrated telephone link 144. The telephone linkage 144 allows data to be directly communicated from the telemetric unit 130 to a suitable data processing unit 145 which can perform data storage and/or analysis off-site from the ambulatory unit 120 and telemetric unit 130.

● Third Embodiment—Space Vehicle Gravida Monitoring System ●

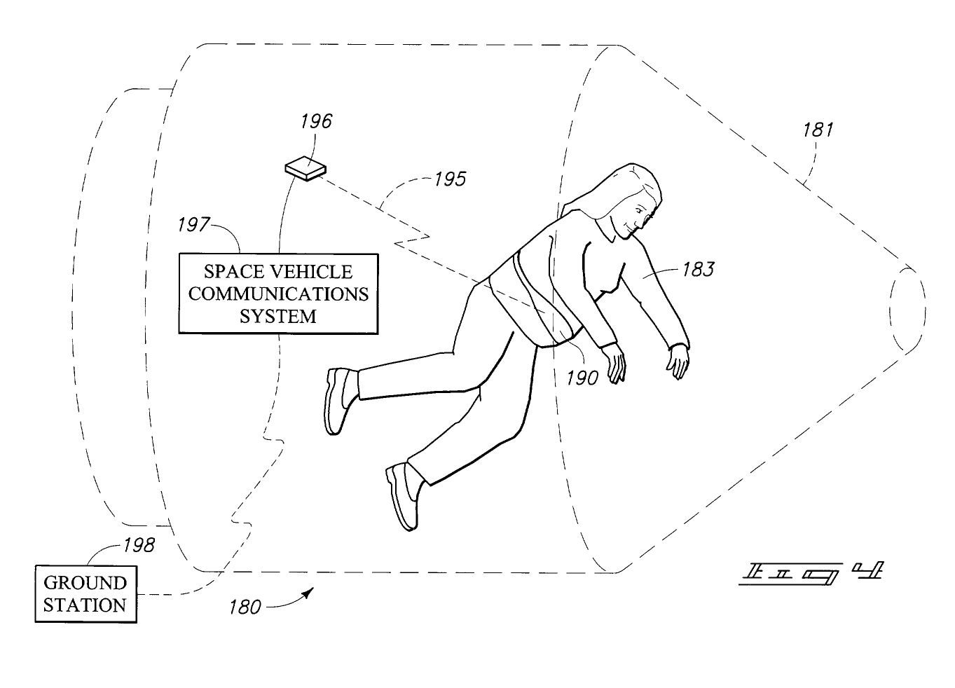

FIG. 4 shows another system 180 having features according to the invention. The system of FIG. 4 is shown in use in a space vehicle 181 to monitor a pregnant woman 183 to indicate situations where the health of the woman's fetus is in need of continuous or periodic monitoring. The space vehicle system includes an ambulatory gravida unit 190 having a sensor support for supporting sensors in a configuration, and can include some or all of the features described above in connection with the first and second systems. The ambulatory unit 190 has a transmitter (not illustrated in FIG. 4) which transmits information telemetrically via a signal 195 to a telemetric receiver unit 196. The telemetric receiver unit 196 is preferably connected to a data processing unit separate from or included as part of vehicle communications system 197. This may be a separate unit or be in the form of a space vehicle general communications system 197 which may relay information to a ground station 198 for data processing, data analysis, medical analysis, or other analysis as desired. It is also possible to relay communications to other parties of interest. Additional possible communications links may also include connection of the ground station directly or indirectly to a global or wide area computer information communications system (not shown in FIG. 4).

● Educational Monitoring System with Mounted Display ●

FIG. 5 shows another system 300 built using at least some of the inventive concepts according to the invention. System 300 of FIG. 5 includes an ambulatory unit 320 installed or otherwise applied to a pregnant woman 311. The system has a telemetric communications capability the same or similar to those described above allowing telemetric one-way or two-way communications between ambulatory unit 320 and telemetric unit 330 forming part of a station 331.

The ambulatory unit 320 communicates with station 331 conveniently mounted for use and observation by woman 311. The telemetric communications unit 330 is incorporated in total or in part within the mounted station 331. Alternatively, telemetric communications unit 330 may be mounted remote from station 331, such as in the near vicinity.

Station unit 331 is also advantageously provided with a mounting arm 346 which allows adjustable positioning relative to a seating chair 340 to which it is mounted.

FIG. 5 shows station 331 having a mounted unit 345 which includes a visual display on the display face directed toward woman 311 for viewing by her. The display on unit 345 may have input and response keys (not shown) which may be soft keys (touch screen), hard keys (not shown) or other input features for receiving commands and operational choices made by woman 311. As shown, the display is preferably a touch screen display that allows a variety of programs and software to be displayed and the woman may respond using touch screen input.

Station unit 345 preferably is capable of propounding questions to woman 311 by display and/or sounds emanating from a speaker 350. Further it is possible to display text, graphs, oscilloscope scans, still images, moving images and a variety of other images and/or other information to woman 311. This can include information being obtained and deduced from information sensed by ambulatory unit 320 either delayed or on a real-time basis.

The station of FIG. 5 also allows the patient to be presented with various educational information of general or more specific types. Software based multiple foreign language versions or translations of presentations will advantageously be made available. In obstetrics, information relating to prenatal care may be suitable for use with some or all pregnant women. It is also possible to present more specific information directed to a particular condition or health risk which she or her fetus are experiencing or which may be otherwise of interest or concern. Still further, it is possible to present programs which her doctor prescribes to make sure training is given in specific areas pertinent to treatments planned or symptoms to be watched. More generalized educational programs concerning such things as prenatal health, general health or other topics may also be presented.

The station of FIG. 5 can still further utilize the display and any touch screen capabilities, input keys or other input devices included with the interface unit 345 to allow woman 311 to answer questions which are saved in the system data processing unit (not shown in FIG. 5). The questions may involve relatively routine questions about the patient such as, name, address, conditions, doctor, medications, etc. Additionally or alternatively, the questions posed to patient 311 may involve testing to determine comprehension of a health training course presented to the patient as she is being monitored for her health signs and/or health signs of her fetus using ambulatory unit 320.

● Educational Monitoring System with Portable Display Communicator ●

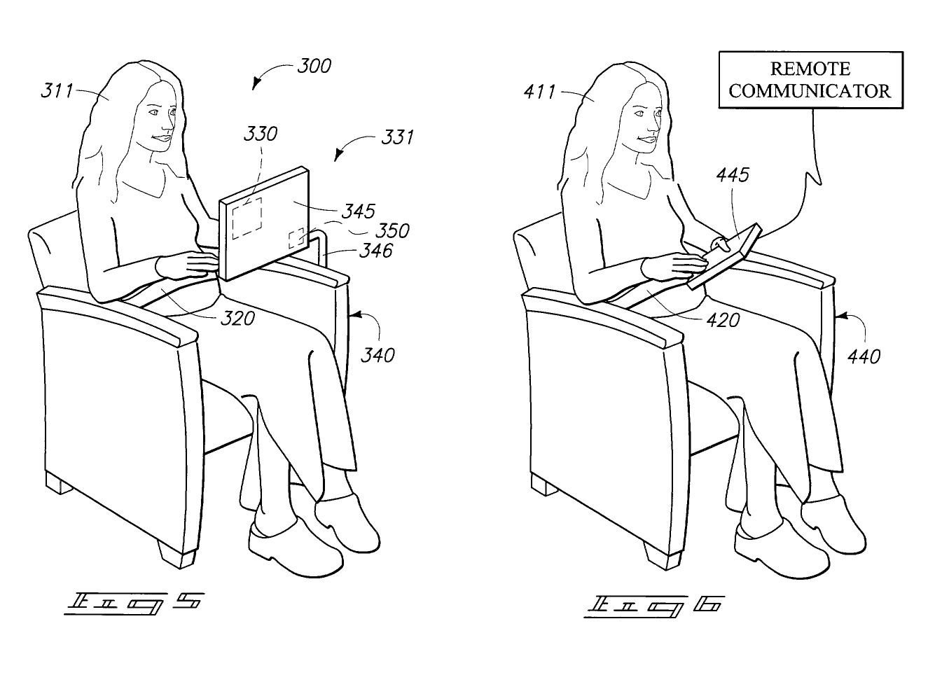

FIG. 6 shows another version of the invention wherein the patient 411 has a gravida unit 420 which may be constructed according to the various descriptions contained elsewhere in this document. The gravida unit is either wired to or telemetrically linked to a portable interface unit 445 similar to unit 345 described herein above in reference to FIG. 5. The description thereof otherwise applies except that unit 445 is not mounted to chair 440. Therefore, such description will not be repeated but fully applies.

● Methods & Operation—Introduction ●

The inventions may also include novel methods, operational features, uses and other aspects. Such methods, operations and uses of the invention may be fully or partly described above. Exemplary methods, operation and additional description will now be given detailing aspects which may be taken alone or in combination with other description given herein to define the inventions.

● Methods for Routine Monitoring of Fetal Well-Being ●

The invention includes methods for routine monitoring of fetal well-being using the systems and techniques described herein. Such methods may advantageously involve applying a gravida unit having the various features or combination of features selected from the preferred and alternative constructions given elsewhere in this document. The gravida unit may be selected to have telemetric signal transmission or signal transmission and receiving capabilities depending upon the abilities desired in the system being employed. Where display of testing information or other information is desired, then the gravida unit or associated interface unit will need to be able to both transmit and receive information to the unit with which it is linked in communication.

The gravida units may also be chosen by selecting a construction which provides hard wiring between the gravida units and an associated interface unit.

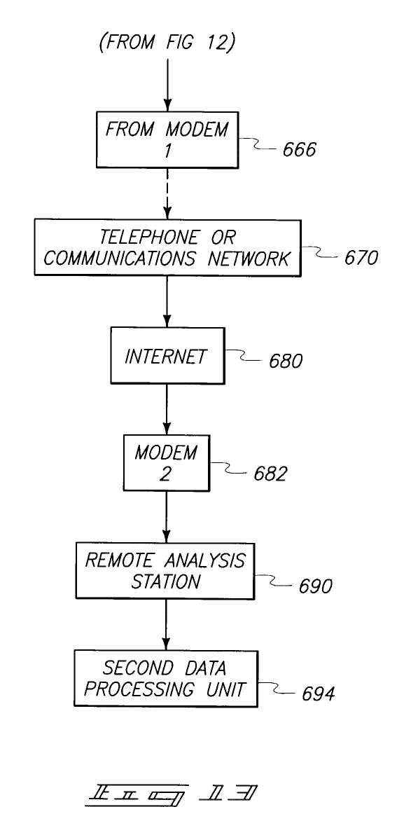

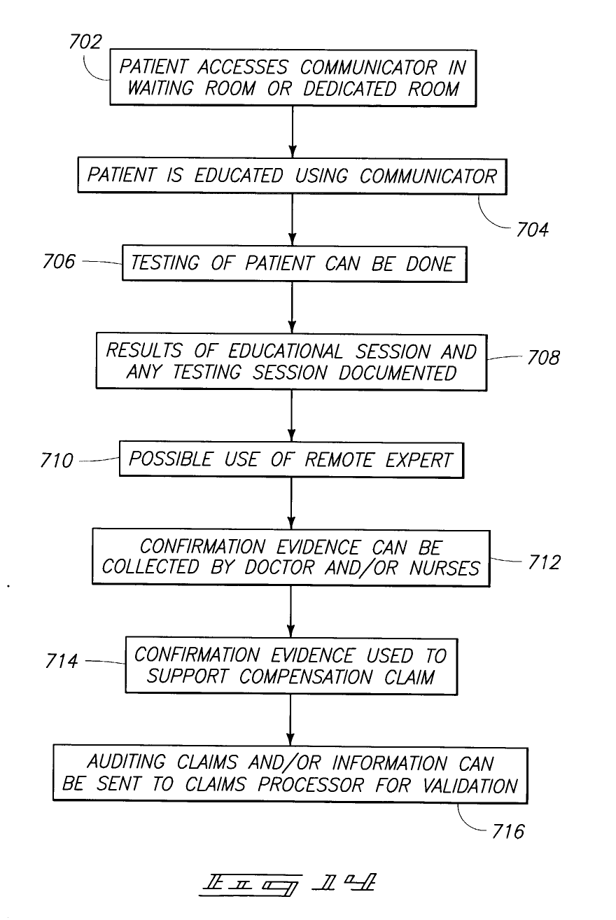

FIGS. 12 and 13 show steps in the processing of preferred signals and communications using the invention. In FIG. 12 the ambulatory gravida unit is represented as it is used in the generating of sensor signals using sensors such as 40, 41, 51, 52 and others described herein. The signal generating step leads to communicating of the sensed signals to ambulatory sensor pre-amplifier circuitry 610 of conventional design which is advantageously carried upon the gravida unit to provide some improvement of the sensor output signals prior to any attempt to further communicate or transmit such signals. The pre-amplification circuitry provides boosted signals which are used by an ambulatory transmitter or transceiver 620 such as described hereinabove. The transmitter or transceiver circuitry can be of various conventional designs for performing the indicated signal transmitting function to a remotely located reception station 630, such as station 35 of FIG. 1 or other similar units for receiving or receiving and sending signals and data therebetween.

The receiving station 630 is preferably connected to a relay station transceiver 640 which relays one-way or two-communications relative to monitoring transceiver or receiver station 630. The relay station 640 may be linked either by wiring or telemetrically to a further or second relay station 650.

The data or signals being communicated and relayed are preferably communicated by communicating between the relay stations 640 and 650 and a data processing unit 660. Data processing unit 660 performs by processing the incoming signals or data and providing signals or data to an associated data processing station display 662 and data storage unit 664. First data processing unit 660 further is advantageously connected to a first modem 666.

FIG. 13 shows that the methods may also employ transmitting data from processing unit 660 via modem 666 to a telephone or other communications network 670. The telephone or other communicating may further be enhanced by connecting the communications via the internet 680 to a second modem 682 which is part of a remote analysis station 690. The remote analysis station 690 includes a remote or second data processing unit 694 which can be used by a remote attending physician to analyze data gathered by the ambulatory gravida unit and perform various additional analysis therefrom.

● Methods for Combined Fetal Monitoring and Prenatal Education and/or Testing ●

Of particular advantage are methods according to the invention which provide for the combined actions of fetal monitoring and gravida education and/or testing in a simultaneous or substantially simultaneous operations. In such methods the fetal sensing, monitoring, and analysis can be performed along with the patient being given an educational vignette as displayed and potentially presented with associated audio information, such as shown in the systems of FIGS. 5 and 6, in particular.

● Methods for Monitoring, Education and Verification ●

Methods according to the invention include the various steps described hereinabove and can additionally be used with confirming of the delivery of medical services and any providing of educational programs or providing of any testing of the gravida or other patient to allow verification by a health provider or funding insurance company or government agency. The methods are illustrated at FIG. 14. The methods include having the patient access the communicator, such as gravida unit 320 or 420 and interface units 345 or 445 as shown in step 702 of FIG. 14. The accessing step allows the patient to be queried on routine data fields, such as name, address, and other identification information.

In one type of preferred methods according to the invention, the patient is educated using the communicator, such as interface units 345 or 445. The educating step is performed by displaying visual information and sounding audio information or both as needed to performing the educating step.

Step 706 of FIG. 14 indicates that the methods may further employ testing or quizzing of the patient. This step may be testing about the educating step and information presented therein. Alternatively, it may be testing about other types of information used to confirm identity or for assessing the knowledge and background of the patient relative to providing medical services or the patient's ability to understand the information that may be presented. Depending on the testing of this type the educating or testing programs may be adjusted by adjusting the difficulty of information being provided or by providing intervening educating steps that further prepare the patient prior to performing any needed testing. Thus the educating and/or testing may be an iterating process.

Step 708 indicates that the methods also preferably include documenting the results of educating and testing. This may be done by recording relevant information from such processes in either paper, electronic or other suitable form. In some forms of the inventive methods, the patient is presented with a paper confirmation report which is acted upon by the patient signing the document attesting to the actual and legitimate performing of the educational or testing services by the doctor, nurse, pharmacist or other health care provider. Thereafter the confirming evidence that medical services have been performed can be collected in step 712. Step 712 may also include testing or quizzing, for example questioning, by the doctor, nurse, pharmacist or other monitor to further confirm that the educating and/or testing done using the automated systems described herein were successfully performed. The doctor, nurse, pharmacist or other health provider may then perform by signing the confirming evidence to attest to the proper delivery of medical services as indicated in the confirmation evidence.

Step 710 indicates that the novel methods may also optionally employ using a remote expert. The employing of a remote expert may involve analyzing the sensed information from the gravida unit, or may involving analyzing or assessing various testing or educating information given to or recorded from the patient. These added factors of analysis may be combined with the analyzing of sensed information from the gravida unit or testing answers to providing diagnosing of patient status and assessing of fetal and maternal well-being.

Step 714 represents supporting a compensation claim made to an insurance company, government health agency or other health care funding agency by providing the confirmation evidence relating to the various service or services which were performed and documented. This can entail providing any needed coding for the type of service involved and automated submission of such claims to the funding agency, by transmitting data files containing such claims.

Step 716 represents processing or auditing claims submitted to the funding organization. This may be done initially in response to the initial claiming of compensation to perform a verifying or validating operation needed prior to payment being made. Alternatively or additively, it may be done in an auditing phase wherein the above-described procedures are used along with the confirmatory evidence indicating provision of the services to provide validation or revalidation that prior payment was properly made in connection with services actually rendered.

● Manner of Making ●

The invention is preferably manufactured or otherwise made using preexisting metal and plastic working techniques, electronics production techniques and other techniques as needed to provide the apparatus and systems described herein. Future techniques hereafter developed for providing the indicated structures and functions are properly employed in making of apparatuses and systems hereunder.

● Further Explanation Concerning Aspects of the Invention ●

The invention has been described with regard to a number of features, steps, functions, processes, methods, uses and other aspects. One or more of these are or may be characteristic of the inventions taught by this disclosure. The language and terminology included in the claims and abstract are also descriptive of the inventions and is incorporated by reference as part of the description of the inventions. Any one or more of the features or aspects described, or any combinations of such features or aspects may be important or significant in defining and characterizing the inventions.

The inventions have been described in compliance with the applicable statutory provisions. Because of the inherent limitations of language, it should be understood that the inventions may include various aspects, features or concepts not necessarily limited to the specific forms and features described and shown herein. The description given shows one or more currently preferred forms for implementing the inventions.

1. A fetal monitoring system, including:
at least one gravida unit adapted for application to a pregnant woman to obtain fetal health information concerning a fetus in utero carried within the pregnant woman;
at least one fetal sensor on said at least one gravida unit for sensing indications of fetal health;
at least one receiving station for telemetrically receiving communications of fetal health information obtained by said at least one gravida unit;
at least one signal transmission device for telemetrically communicating said communications of fetal health information originating from the at least one gravida unit to said at least one receiving station to provide communications therebetween;
said at least one receiving station being physically disconnected from said at least one gravida contact unit to allow untethered movement of a pregnant women using the gravida unit relative to the receiving station.

2. The fetal monitoring system of claim 1 wherein said at least one fetal sensor includes a plurality of fetal sensors.

3. The fetal monitoring system of claim 1 wherein said at least one fetal sensor includes at least one fetal cardiac sensor.

4. The fetal monitoring system of claim 1 wherein said at least one fetal sensor includes at least one acoustic fetal sensor.

5. The fetal monitoring system of claim 1 wherein said at least one fetal sensor includes at least one fetal movement sensor.

6. The fetal monitoring system of claim 1 further including an interferometer for comparing aspects of at least one fetal sensor signal to provide an indication of fetal health.

7. The fetal monitoring system of claim 1, and further including:
at least one relay station which receives communications of fetal health information originating from the at least one gravida contact unit;
a base station for receiving the fetal health information from the at least one relay station.

8. The fetal monitoring system of claim 1, and further including:
at least one relay station which receives communications of fetal health information originating from the at least one gravida contact unit;
a base station for receiving the fetal health information from the at least one relay station;
and further defined by the base station being on a satellite.

9. The fetal monitoring system of claim 1, and further including:
at least one relay station which receives communications of fetal health information originating from the at least one gravida unit;
a base station for receiving the fetal health information from the at least one relay station;
and further defined by the base station being on a manned spacecraft.

10. The fetal monitoring system of claim 1 wherein said receiving station is upon a satellite.

11. The fetal monitoring system of claim 1 wherein said receiving station is upon a spacecraft.

12. The fetal monitoring system of claim 1 wherein said signal transmission device is on said at least one gravida contact unit.

13. The fetal monitoring system of claim 1 wherein said at least one receiving station is connected to the internet to communicate said fetal health information for review by a medical expert at a remote location.

14. The fetal monitoring system of claim 1 and further including at least one relay communicator which receives the fetal health information and relays it to said at least one receiving station.

15. The fetal monitoring system of claim 1 and further including at least one relay communicator which receives the fetal health information and relays it to said at least one receiving station.

16. The fetal monitoring system of claim 1 and further including at least one relay communicator which receives the fetal health information and relays it to said receiving station, said relay communicator having a visual display for providing information to the patient.

17. The fetal monitoring system of claim 1 and further including at least one motion detector for detecting fetal movement in utero.

18. The fetal monitoring system of claim 1 and further including at least one manual motion sensor which is activated by the pregnant woman when fetal movement in utero is noticed by the pregnant woman.

19. The fetal monitoring system of claim 1 and further including at least one disablement sensor which senses when the pregnant woman wishes to disable the fetal monitoring system, such as when the pregnant woman is planning to undertake activity which would adversely affect the fetal monitoring results.

20. The fetal monitoring system of claim 1 wherein the at least one gravida contact unit includes mounting portions which are adapted to extend about the pregnant woman's torso when used thereon.

21. The fetal monitoring system of claim 1 wherein the at least one gravida contact unit includes:
mounting portions which are adapted to extend circumferentially about the pregnant woman's torso when used thereon;
a clasp which is capable of connecting and disconnecting said mounting portions to apply the at least one gravida unit against the pregnant woman.

22. A method for collecting fetal health information, including:
applying at least one gravida unit to a pregnant woman, said at least one gravida unit having at least one fetal sensor for sensing indications of fetal health;
sensing indications of fetal health using said at least one fetal sensor for a fetus in utero carried within the pregnant woman;
transmitting fetal health information from said at least one gravida unit using at least one telemetric transmission device to at least one receiving station adapted to receive such fetal health information using a telemetric communications linkage which provides communications contact therebetween;
receiving said fetal health information at said at least one receiving station;
analyzing the fetal health information received by the at least one receiving station to indicate fetal health.

23. A method according to claim 22 and further including recording the fetal health information received by the at least one receiving station.

24. A method according to claim 22 and further including relaying fetal health information between the gravida unit and a relaying transceiver.

25. A method according to claim 22,
further including relaying fetal health information sensed in said sensing step before such fetal health information is received by the at least one receiving station;
wherein said receiving said fetal health information is performed by at least one receiving station which is located in a space vehicle.

26. A method according to claim 22 wherein said sensing indications of fetal health includes sensing fetal cardiac information.

27. A method according to claim 22 wherein said sensing indications of fetal health includes sensing acoustic fetal information.

28. A method according to claim 22 wherein said sensing indications of fetal health includes sensing acoustic fetal information.

29. A method according to claim 22 wherein said at least one fetal sensor includes a plurality of fetal sensors.

30. A method according to claim 22 wherein said at least one fetal sensor includes a plurality of fetal sensors, and said plurality of fetal sensors emit fetal sensor signals which are affected by a fetus in utero during transmission between the plurality of fetal sensors and said receiving station to thereby indicate fetal health.

31. A method according to claim 22 wherein said at least one fetal sensor includes a plurality of fetal sensors and said plurality of fetal sensors emit fetal sensor signals which indicate fetal health by phase shift between the fetal sensor signals emitted from the plurality of fetal sensors.

32. A method according to claim 22 wherein said at least one fetal sensor includes at least one fetal cardiac sensor.

33. A method according to claim 22 wherein said at least one fetal sensor includes at least one inductive fetal sensor.

34. A method according to claim 22 wherein said at least one fetal sensor includes at least one acoustic fetal sensor.

35. A method according to claim 22 wherein said at least one fetal sensor includes at least one fetal movement sensor.

36. A method according to claim 22 wherein said receiving station is upon a space vehicle.

37. A method according to claim 22 wherein said receiving station is upon a satellite.

38. A method according to claim 22 wherein said signal transmission device is on said at least one gravida unit.

39. A method according to claim 22 wherein said at least one receiving station is connected to the internet to communicate said fetal health information for review by a medical expert at a remote location from the at least one gravida unit.

40. A method according to claim 22 wherein and further including at least one relay communicator which receives the fetal health information and relays it to said at least one receiving station.

41. A method according to claim 22 wherein and further including at least one relay communicator which includes said signal transmission device, and which receives the fetal health information and relays it to said at least one receiving station.

42. A method according to claim 22 wherein and further including at least one relay communicator which includes said signal transmission device, and receives the fetal health information and relays it to said receiving station, said relay communicator having a visual display for providing information.

43. A method according to claim 22 wherein and further including at least one motion detector for detecting fetal movement in utero.

44. A method according to claim 22 wherein and further including at least one manual motion sensor which is activated by the pregnant woman when fetal movement in utero is noticed by the pregnant woman.

45. A method according to claim 22 wherein the at least one gravida unit includes mounting portions which are adapted to extend about the pregnant woman's torso when used thereon.

46. A method according to claim 22 wherein the at least one gravida unit includes:
mounting portions which are sufficiently sized and adapted to extend circumferentially about the pregnant woman's torso when used thereon;
a clasp for controlled connection and disconnection of the at least one gravida unit.

47. A method for health care delivery and monitoring, including:
receiving a gravida at a reception station having an associated reception area;
providing the gravida with a diagnostic testing unit to be used by the patient to test fetal well-being;
having the gravida apply the diagnostic testing unit in position to conduct testing on the gravida;
performing diagnostic fetal testing using the testing unit as applied by the gravida;
recording information indicating results of the diagnostic fetal testing;
conducting a personal examination using a health care professional who meets with the gravida concerning the diagnostic fetal testing to analyze and confirm results of the diagnostic fetal testing.

48. A method according to claim 47 and further including providing an educational program to the gravida during the testing on the gravida.

49. A method according to claim 47 and further including providing a personalized educational program to the gravida during the testing on the gravida.

50. A method according to claim 47 and further including providing an educational program to the gravida during the testing on the gravida.

51. A method according to claim 47 and further including:
providing an educational program to the gravida during the testing on the gravida;
quizzing the gravida to determine retention of lessons provided during the educational program.

52. A method according to claim 47 and further including:
providing an educational program to the gravida during the testing on the gravida;
quizzing the gravida to determine retention of lessons provided during the educational program;
recording quiz results from said quizzing step.

53. A method according to claim 47 and further including:
providing an educational program to the gravida during the testing on the gravida;
quizzing the gravida to determine retention of lessons provided during the educational program;
recording quiz results from said quizzing step;
questioning the gravida during the personal examination concerning quiz results or lessons provided during the educational program.

54. A method according to claim 47 and further including:
providing an educational program to the gravida during the testing on the gravida;
quizzing the gravida to determine retention of lessons provided during the educational program;
recording quiz results from said quizzing step;
questioning the gravida during the personal examination concerning quiz results or lessons provided during the educational program.

55. A method for health care delivery and monitoring, comprising:
receiving a patient at a reception station having an associated reception area;
providing the patient with a diagnostic testing unit to be used by the patient;
having the patient apply the diagnostic testing unit in position to conduct testing on the patient;
performing diagnostic testing using the testing unit as applied by the patent;
recording information indicating results of the diagnostic testing;
conducting a personal examination using a health care professional who meets with the patient concerning the diagnostic testing to analyze and confirm results of the diagnostic testing.

56. A method according to claim 55 and further including providing an educational program to the patient during the testing on the patient.

57. A method according to claim 55 and further including providing a personalized educational program to the patient during the testing on the patient.

58. A method according to claim 55 and further including providing an educational program to the patient during the testing on the patient.

59. A method according to claim 55 and further including:
providing an educational program to the patient during the testing on the patient;
quizzing the patient to determine retention of lessons provided during the educational program.

60. A method according to claim 55 and further including:
providing an educational program to the patient during the testing on the patient;
quizzing the patient to determine retention of lessons provided during the educational program;
recording quiz results from said quizzing step.

61. A method according to claim 55 and further including:
providing an educational program to the patient during the testing on the patient;
quizzing the patient to determine retention of lessons provided during the educational program;
recording quiz results from said quizzing step;
questioning the patient during the personal examination concerning quiz results or lessons provided during the educational program.

62. A method according to claim 55 and further including:
providing an educational program to the patient during the testing on the patient;
quizzing the patient to determine retention of lessons provided during the educational program;
recording quiz results from said quizzing step;
questioning the patient during the personal examination concerning quiz results or lessons provided during the educational program.
